(19) United States
(12) Plant Patent Application Publication
(10) Pub. No.: US 2012/0311749 P1
(43) Pub. Date: Dec. 6, 2012

(54) Strawberry plant named 'Cristina'

(50) ;

(75) Inventors: Bruno Mezzetti, Ancona (IT); Franco Capocasa, Massignano-Ascoli Piceno (IT)

(73) Assignee: HARGREAVES PLANTS LIMITED, Spalding (GB)

(21) Appl. No.: 13/506,965

(22) Filed: May 29, 2012

(30) Foreign Application Priority Data
May 30, 2011 (EP) 20111274

(51) Int. Cl. A01H 5/00

(52) U.S. Cl. PLT/208

(57) ABSTRACT

A new and distinct variety of strawberry plant named ‘Cristina’ particularly characterized by high adaptability to non-fumigated soil, very late ripening, very large fruit with good taste, and high productivity, is disclosed.

### CROSS REFERENCE TO RELATED APPLICATIONS

This application claims the benefit of priority from EU Community Plant Variety Rights (CPVR) application no. 20111274, filed May 30, 2011, which is incorporated herein by reference in its entirety.

### GENUS AND SPECIES

Fragaria×ananassa

### VARIETY DENOMINATION

‘Cristina’

### BACKGROUND OF THE NEW PLANT

The present invention relates to a new and distinct June-bearing strawberry variety designated ‘Cristina’ and botanically known as Fragaria×ananassa. This new strawberry variety was discovered in Cesena, Italy in 2001 and originated from a cross between the female parent, selection ‘CN95,602,8’ (unpatented, selection of National Breeding Programme Strawberry Project—Italy) and the male parent, selection ‘CN95,419,4’ (unpatented, selection of National Breeding Programme Strawberry Project—Italy). ‘Cristina’ was first fruited at the ‘P. Rosati’ Experimental Farm of Universitá Politecnica delle Marche in Ancona, Italy in 2003, where it was selected and originally designated AN01,211,51.

The present invention has been found to retain its distinctive characteristics through successive asexual propagations via stolons and tissue culture.

Plant Breeder's Rights for this variety have been applied for in Europe on May 30, 2011 having CPVR application no. 20111274. ‘Cristina’ has not been made publicly available or sold more than one year prior to the filing date of this application.

### SUMMARY OF THE INVENTION

The following are the most outstanding and distinguishing characteristics of this new cultivar when grown under normal horticultural practices in Ancona, Italy.

1. High adaptability to non-fumigated soil;

2. Very late ripening;

3. Very large fruit with good taste; and

4. High productivity.

### DESCRIPTION OF THE PHOTOGRAPHS

The accompanying color photographs show typical specimens of the new variety at various stages of development. The colors shown are as true as can be reasonably obtained by conventional photographic procedures. The photographs were taken from ten-month-old plants.

FIG. 1 shows the whole fruit.

FIG. 2 shows upper and lower surfaces of the leaves of the plant with three leaflets.

FIG. 3 shows overall plant habit including fruit at various stages of development.

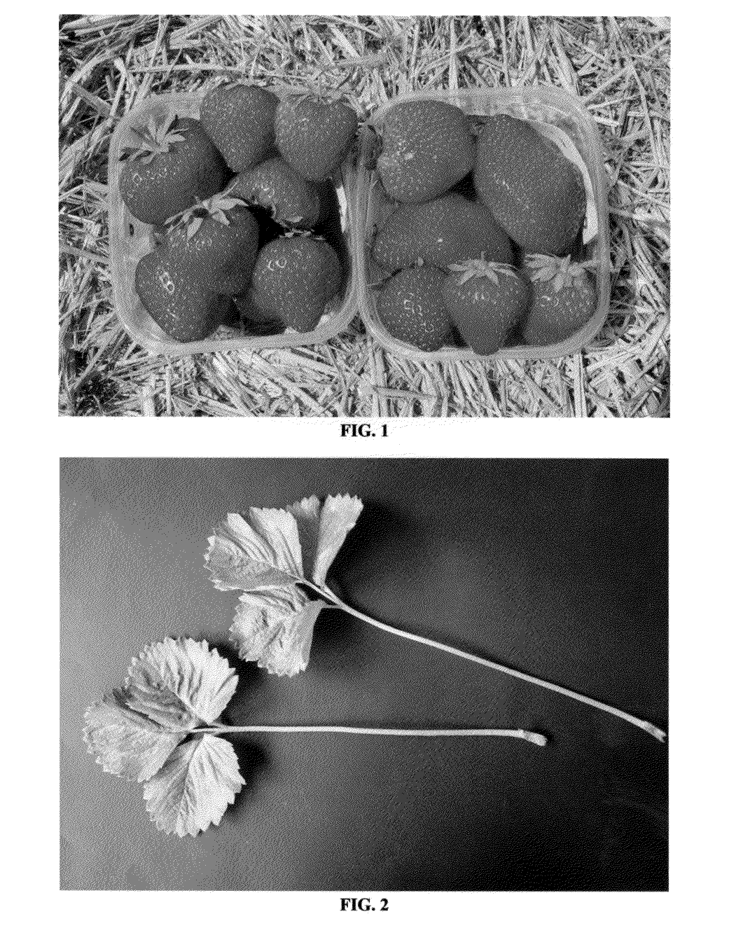

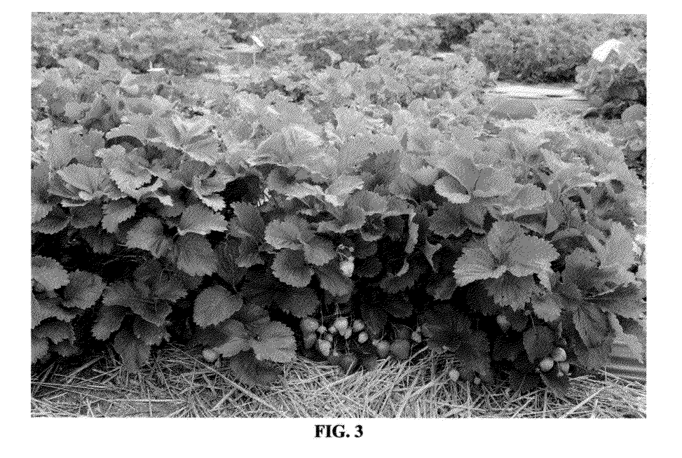

### DESCRIPTION OF THE NEW VARIETY

The following detailed descriptions set forth the distinctive characteristics of ‘Cristina’. The data which define these characteristics is based on observations taken in Ancona, Italy in 2009-2012. ‘Cristina’ was evaluated on non-fumigated soil having the following characteristics: pH 7.9, active calcium 9%, and texture composed at 40% clay, 25% sand and 35% silt. This description is in accordance with UPOV terminology. Color designations, color descriptions, and other phenotypical descriptions may deviate from the stated values and descriptions depending upon variation in environmental, seasonal, climatic, and cultural conditions. ‘Cristina’ has not been observed under all possible environmental conditions. The botanical description of ‘Cristina’ was taken from ten-month-old plants. Color references are primarily to the R.H.S. Colour Chart of The Royal Horticultural Society of London (R.H.S.) and to CIELAB, which is an international color system known as ‘Commission Intenationale De L'Eclairage’, 1978. Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Family.—Rosaceae.
   Species.—Fragaria×ananassa.
   Common name.—Strawberry.
   Denomination.—‘Cristina’.
Parentage:
   Female parent.—‘CN95,602,8’ (unpatented, selection of National Breeding Programme Strawberry Project — Italy).
   Male parent.—‘CN95,419,4’ (unpatented, selection of National Breeding Programme Strawberry Project — Italy).
Plant:
   Growth habit.—Semi-upright.
   Height.—41.9 cm.
   Diameter.—35.5 cm.
   Number of crowns/plant.—3.9.
Terminal leaflets:
   Size.—Length: 10.0 cm Width: 9.8 cm Length/width ratio: 1.02.
   Number of teeth/terminal leaflet.—73.8.
   Shape of teeth.—Rounded.
   Color.—Upper surface: CIELAB L* 37.65, a*−13.91, b* 22.23 Lower surface: CIELAB L* 52.42, a*−11.17, b* 20.37.
   Shape in cross section.—Concave.
   Blistering.—Medium.
   Glossiness.—Medium.
   Number of leaflets.—3.3.
   Base shape.—Obtuse.
   Apex descriptor.—Rounded.
   Pubescence density.—Moderate.
   Margin.—Serrate to crenate.
   Texture.—Medium.
   Venation pattern.—Pinnate Color: Upper surface: RHS 149C Lower surface: RHS 149D.
Petiole:
   Length.—29.2 cm.
   Diameter.—0.39 cm.
   Pubescence density.—Moderate.
   Pose of hairs.—Upwards.
   Color.—RHS 149C.
   Texture.—Medium.
Petiolule:
   Length.—2.74 cm.
   Diameter.—0.19 cm.
   Color.—RHS 149C.
Stipule:
   Length.—3.01 cm.
   Width.—0.75 cm.
   Texture.—Medium.
   Anthocyanin.—Medium.
   Color.—RHS 150C.
Stolon:
   Diameter at bract.—0.20 cm.
   Avg. number of daughter plants.—33.
   Anthocyanin.—Medium.
   Thickness.—Medium.
   Pubescence.—Medium.
Inflorescence:
   Position relative to foliage.—Beneath.
   Time of flowering (50% of plants at first flower).—March 30.
   Corolla.—Shape: Rounded Length: 3.35 cm Width: 3.47 cm.
   Petals.—Shape: Elliptic Apex: Truncate to slightly obtuse Base: Attenuate Margin: Entire Spacing: Touching Length: 1.35 cm Width: 3.03 cm Length/width ratio: 0.44 Color of upper side: White Texture: Medium.
   Calyx.—Diameter: 3.88 cm Diameter relative to corolla: Smaller Inner calyx diameter relative to outer: Same size.
   Sepal.—Margin: Entire, some are jagged Length: 1.32 cm Width: 0.42 cm Typical and observed sepal number per flower: 12.9 Color: RHS 144C.
   Peduncle.—Length: 13.94 cm Diameter: 0.32 cm Color: RHS 149D.
Fruit:
   Size.—Very large.
   Length.—4.44 cm.
   Width.—3.72 cm.
   Length/width ratio.—1.19 (Longer than broad).
   Fruit hollow center (cavity).—Medium.
   Weight (per individual berry).—33.7 g.
   Predominant fruit shape.—Conical.
   Difference in shape between primary and secondary fruits.—Primary fruits are very much larger than secondary fruits.
   Fruit skin color.—RHS 30A.
   Evenness of fruit color.—Even or very slightly uneven.
   Fruit glossiness.—Medium to strong.
   Attitude of calyx segments.—Downwards, some outwards.
   Achenes.—Insertion of achenes: Level with surface Color: Yellow Width of band without achenes: Absent or very narrow.
   Firmness of flesh (when fully ripe).—Firm; 328 g (as detected by penetrometer with 6 mm probe).
   Firmness of skin.—Medium.
   Internal color.—RHS 32D.
   Sweetness.—Medium high; 7.1° Brix.
   Acidity.—Medium; 10.8 meq NaOH/100g.
   Type of bearing.—Not remontant; June-bearing.
   Grams of fruit/plant.—1213.0 g.
   Time of ripening (50% of plants with first ripe fruit).—Very late; May 21.
   Yield.—Medium high.
   Disease, pest, and stress resistance.—Not available.

### COMPARISON WITH PARENTAL AND COMMERCIAL VARIETIES

When ‘Cristina’ is compared to the female parent ‘CN95,602,8’ (unpatented, selection of National Breeding Programme Strawberry Project—Italy), ‘Cristina’ has higher productivity, larger fruit size and later production, whereas ‘CN95,602,8’ has lower productivity, smaller fruit size and earlier production.

When ‘Cristina’ is compared to the male parent ‘CN95,419,4’ (unpatented, selection of National Breeding Programme Strawberry Project—Italy), ‘Cristina’ has higher productivity, larger fruit size and higher fruit firmness, whereas ‘CN95,419,4’ has lower productivity, smaller fruit size and lower fruit firmness.

When ‘Cristina’ is compared to the commercial variety ‘Sonata’ (U.S. Plant Pat. No. 18,000), ‘Cristina' has higher productivity, larger fruit size and later production, whereas ‘Sonata’ has lower productivity, smaller fruit size and earlier production.

We claim:

1. A new and distinct variety of strawberry plant named ‘Cristina’ as described and shown herein.
